(19) United States
(12) Patent Application Publication
(10) Pub. No.: US 2009/0088602 A1
(43) Pub. Date: Apr. 2, 2009

(54) SHEATH STRUCTURE OF PORTABLE MEDICAL EQUIPMENT

(76) Inventor: Donny CHUNG, Taipei (TW)

Correspondence Address: HDLS Patent & Trademark Services, P.O. BOX 220746, CHANTILLY, VA, 20153-0746, US

(21) Appl. No.: 11/865,854

(22) Filed: Oct. 2, 2007

(51) Int. Cl. A61B 1/00

(52) U.S. Cl. 600/125

(57) ABSTRACT

A sheath structure of portable medical equipment includes a protecting shell, a snap-on body, a container, and a sheath. The protecting shell has two shell bodies corresponding to each other. At one side of the shell bodies, a first snapping part and a second snapping part are arranged, and a jointing hole is arranged at the front side of the second snapping part. The snap-on body has a main body jointed to the jointing hole and arranged a ring groove at the front side thereof. The container includes a first cup and a second cup. An opening is respectively arranged at one side of a first bulger and a second bulger, each of which is belonged to the first and second cups, while a projecting wall is respectively arranged at another side. When the first cup and the second cup have been assembled, an accommodating space is formed between the first bulger and the second bulger, and the sheath is contained in the accommodating space with one side positioned in the ring groove. After the snap-on body is snapped on an adapter of the medical equipment, the container is pushed toward a grip structure of the medical equipment, and the sheath is then released out and fitted onto the grip structure of the medical equipment.

### BACKGROUND OF THE INVENTION

1. Field of the Invention

The present invention generally relates to medical equipment, in particular, to a sheath of portable medical equipment.

2. Description of Prior Art

Since medical equipment is a combination of advanced technologies of micro-electro-mechanical technique, communication technique, materials technique and biochemical technique with an integration of indigenously industrial technique, the development of each technique or technology all can actuate the industrial development of medical equipment. Besides, medical equipment with good quality may promote the successful rate of medical care or operation.

A traditionally portable flashlight used in medical care has a grip 10, the inside of which is arranged battery 20, and the front side of which is assembled a tube 30, the interior of the front side of which is disposed a lighting source 40, which is electrically connected to the battery 20 by means of a power transmitting wire 50 and a switch 60. When the equipment is to be operated, the medical personnel just needs to push down the switch 60 to transmit power from the battery 20 through the transmitting wire 50 to the lighting source 40 to light up the lighting source 40, so the medical personnel can clearly examine the wounds of a patient.

When a portable medical equipment of prior arts is being used by medical personnel, there is no any protecting or waterproofing design in the portable medical equipment, so the blood of the patient easily filtrates into the portable medical equipment, making it electrical shortcut or rusty. In a more serious case, bacteria will even reside on the portable medical equipment.

Accordingly, aiming to solve aforementioned shortcomings, after a substantially devoted study, in cooperation with the application of relatively academic principles, the inventor finally proposes the present invention that is designed reasonably to possess the capability to improve the prior arts significantly.

### SUMMARY OF THE INVENTION

The invention is mainly to provide a sheath structure fitted over portable medical equipment to prevent the equipment from being stained, infiltrated or infected by the patient's blood.

The invention is to provide a sheath structure of portable medical equipment, which includes: a protecting shell, a snap-on body, a container, and a sheath. The protecting shell has two shell bodies corresponding to each other. At one side of the shell bodies, there is arranged a first snapping part, on the surface of which a snapping face is arranged. At front side of the first snapping part, a second snapping part with a snapping rib is connected, and at front side of the second snapping part, a jointing hole communicating the interior of the protecting shell is arranged. The snap-on body has a main body, two sides of which are connected two catches corresponding to each other, and through which a perforation is arranged, and between the perforation and the catches, a ring groove is arranged for snapping on one side of the sheath. The container includes a first cup and a second cup. A first bulger is arranged at the first cup and has a snapping side at one side thereof. An opening is arranged at the snapping side for providing the second snapping part of the protecting shell to pass through, making the snapping side and the snapping rib of the second snapping part snapped together. In addition, a first projecting wall is extended at another side of the first bulger. The second cup has a second bulger having an inner diameter larger than an outer diameter of the first bulger. An opening is arranged on a side face of the second bulger for providing the second snapping part to pass through. Furthermore, a second projecting wall is extended at another side of the second bulger. During assembly, an accommodating space is formed between the first bulger of the first cup and the second bulger of the second cup for accommodating the sheath, at which a first side and a second side are arranged. A hole arranged on the side face of the first side is disposed in the ring groove of the snap-on body, while the side face of the second side is shown as an opening. After the sheath between the first side and the second side has been folded, the opening of the second side is fitted over the bulger of the first cup, making the folded sheath accommodated in the accommodating space.

### BRIEF DESCRIPTION OF DRAWING

The features of the invention believed to be novel are set forth with particularity in the appended claims. The invention itself, however, may be best understood by reference to the following detailed description of the invention, which describes a number of exemplary embodiments of the invention, taken in conjunction with the accompanying drawings, in which:

FIG. 1 is an illustration of the outer appearance of traditional portable medical equipment;

FIG. 2 is a perspective illustration of the outer appearance of a sheath structure of the present invention;

FIG. 3 is an explosive illustration of a sheath structure of the present invention;

FIG. 4 to FIG. 6 are executing illustrations of a sheath structure of the present invention, which is being fitted over a portable medical equipment;

FIG. 7 is an illustration of another embodiment of the present invention; and

FIG. 8 is an illustration of a further embodiment of the present invention.

### DETAILED DESCRIPTION OF THE INVENTION

In cooperation with attached drawings, the technical contents and detailed description of the present invention are described thereinafter according to a number of preferable embodiments, being not used to limit its executing scope. Any equivalent variation and modification made according to appended claims is all covered by the claims claimed by the present invention.

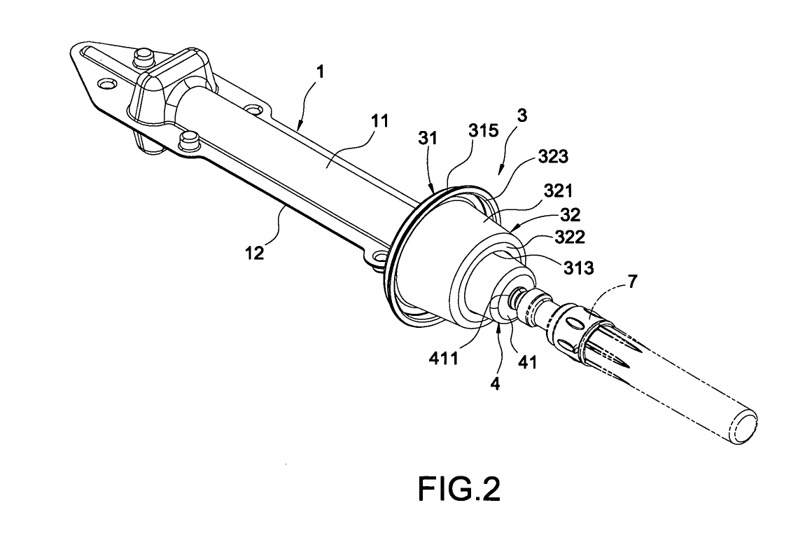

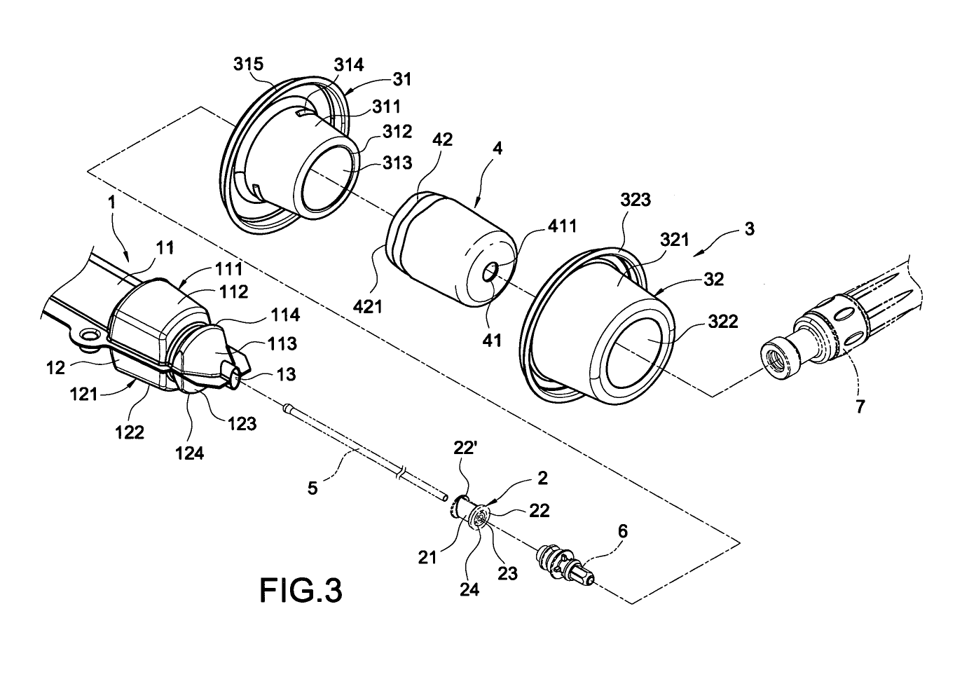

Please refer to FIG. 2 to FIG. 4, which respectively are a perspective, explosive, and cross-sectional illustrations of a sheath structure of the present invention. As shown in these figures, the sheath structure of portable medical equipment according to the present invention includes: a protecting shell 1, a snap-on body 2, a container 3, and a sheath 4.

The protecting shell 1 is a hollow body made of transparent plastic materials for packaging a light source transmitting body (i.e. optical fiber) 5 of the medical equipment and includes two shell bodies 11, 12 corresponding to each other. Two first snapping parts 111, 121 shown as projecting shape are arranged at one side of two shell bodies 11, 12. Two snapping faces 112, 122, shown as arc shape and snapped tightly in the container 3, are arranged on the surface of the first snapping part 111. Two second snapping parts 113, 123 shown as cone shape are connected to the front side of the first snapping parts 111, 121. Two snapping ribs 114, 124 are arranged on the second snapping parts 113, 123 for being snapped in the container 3. In addition, a jointing hole 13 communicating the interior of the protecting shell 1 is arranged at the front side of the second snapping parts 113, 123 for jointing the snap-on body 2.

The snap-on body 2 shown as an “I” shape has a main body 21 assembled to the jointing hole 13. Two catches 22, 22′ corresponding to each other are connected to the front and rear sides of the main body 21. Furthermore, a perforation 23 is arranged at the main body 21 for providing a light source transmitting body 5 to pass through. On the other hand, a ring groove 24 is arranged between the perforation 23 and the catch 22 for providing one side of the sheath 4 to be snapped on.

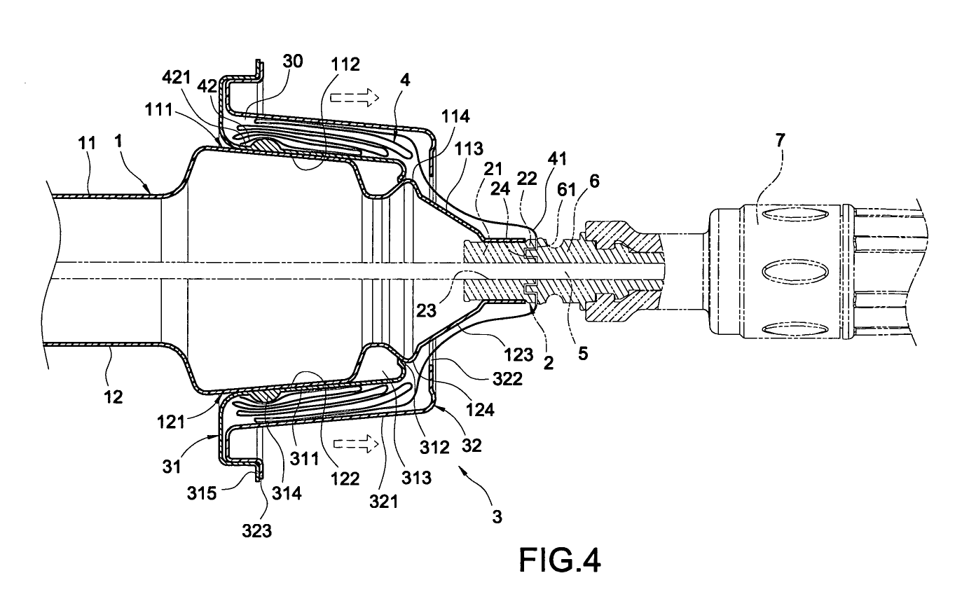

The container 3 includes a first cup 31 and a second cup 32. The first cup 31 has a hollow first bulger 311, one side of which is arranged a snapping side 312 curved inwardly, on which an opening 313 is arranged for providing the second snapping parts 113, 123 of the protecting shell 1 to pass through, making the snapping 312 and the second snapping parts 113, 123 snapped together. In addition, a rib ring 314 is arranged on the outer surface of the first bulger 311 for snapping the folded sheath 4. A first projecting wall 315 folded outwardly is extended at another side of the first bulger 311. The second cup 32 has a second bulger 321 having an inner diameter larger than an outer diameter of the first bulger 311 of the first cup 31. An opening 322 is arranged on one side face of the second bulger 321 for providing the second snapping part 13 to pass through. In addition, a second projecting wall 323 folded outwardly is extended at another side of the second bulger 321. When the first cup 31 and the second cup 32 are jointed together, the second projecting wall 323 of the second cup 32 is embedded between the inner wall surface of the first projecting wall 315 of the first cup 31 and the first bulger 311, making an accommodating space 30 formed between the outer surface of the first bulger 311 of the first cup 31 and the inner surface of the second bulger 321 of the second cup 32 for accommodating the sheath 4 (please refer to FIG. 4).

The sheath 4, a thin film envelope made of transparent plastic, has a first side 41 and a second side. A hole 411 arranged on the side face of the first side 41 is disposed in the ring groove 24 of the snap-on body 2, while the side face of the second side 42 is shown as an opening 421. After the sheath 4 between the first side 41 and the second side 42 is folded, the opening of the second 42 is fitted over the outside of the first bulger 311 of the first cup 31, making the folded sheath 4 accommodated in the accommodating space 30.

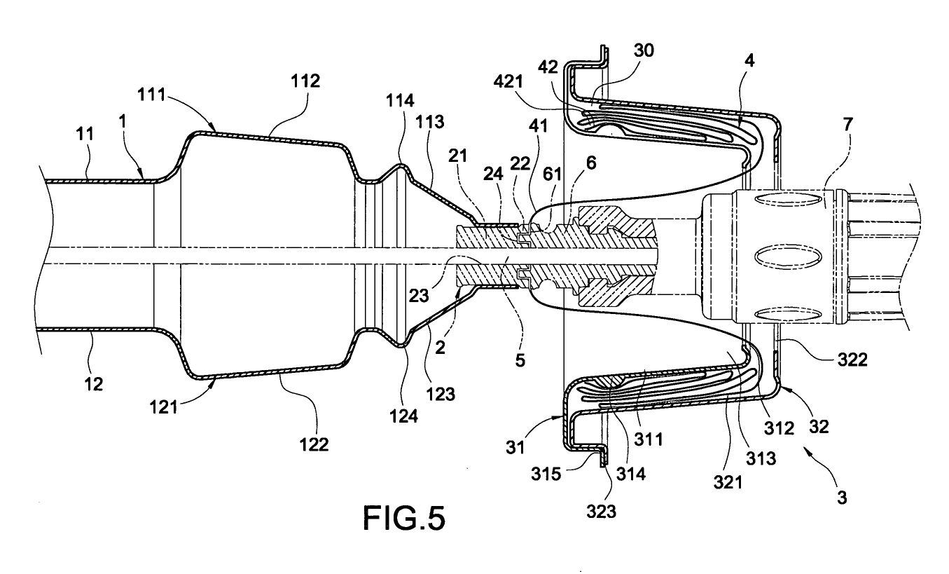

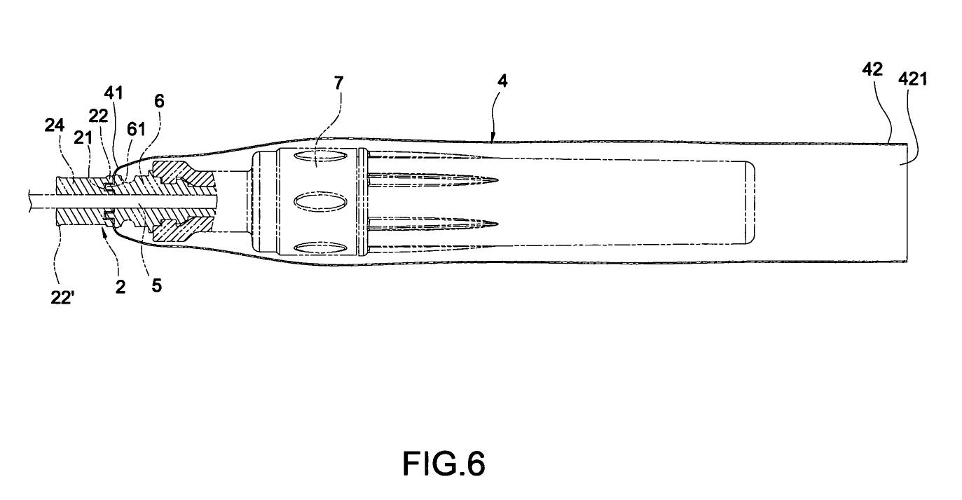

Please refer to FIG. 4 to FIG. 6, which are executing illustrations of a sheath structure of the present invention, which is being fitted over portable medical equipment. As shown in these figures, before the sheath structure of the present invention is applied to portable medical equipment, the ring groove 24 at one side of the snap-on body 2 is first snapped on a projecting ring 61 at front side of the adapter 6, making the hole 411 of the first side 41 of the sheath 4 snapped on between the snap-on body 2 and adapter 6. Then, the container 3 is pushed toward the grip structure 7, making the container 3 separated from the first snapping parts 111, 121 and the second snapping parts 113, 123 at the front side of the sheath 1. Finally, the sheath 4 accommodated between the first bulger 311 of the first cup 31 and the second bulger 321 of the second cup 32 is released in sequence and covers the entire grip structure 7, thus it being possible to keep the grip structure 7 from being infected by the blood or the bacteria, during a medical care or operation. After use, the snap-on body 2 is separated from the adapter 6, the sheath 4 being taken down and thrown away.

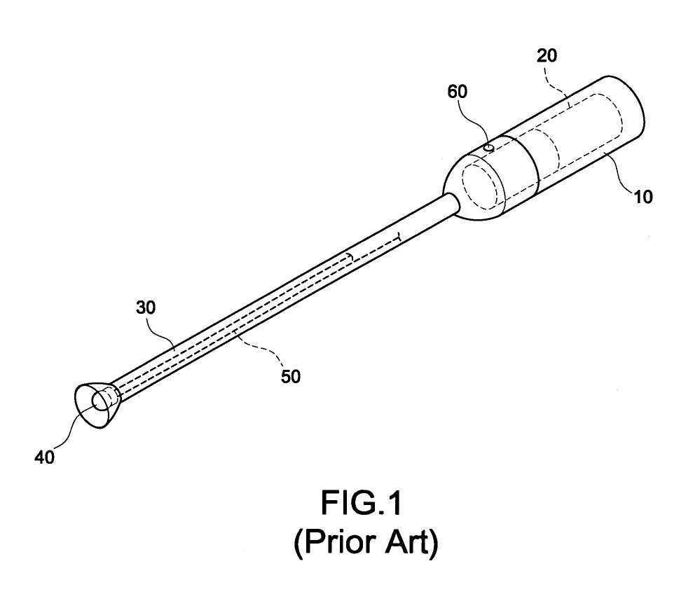

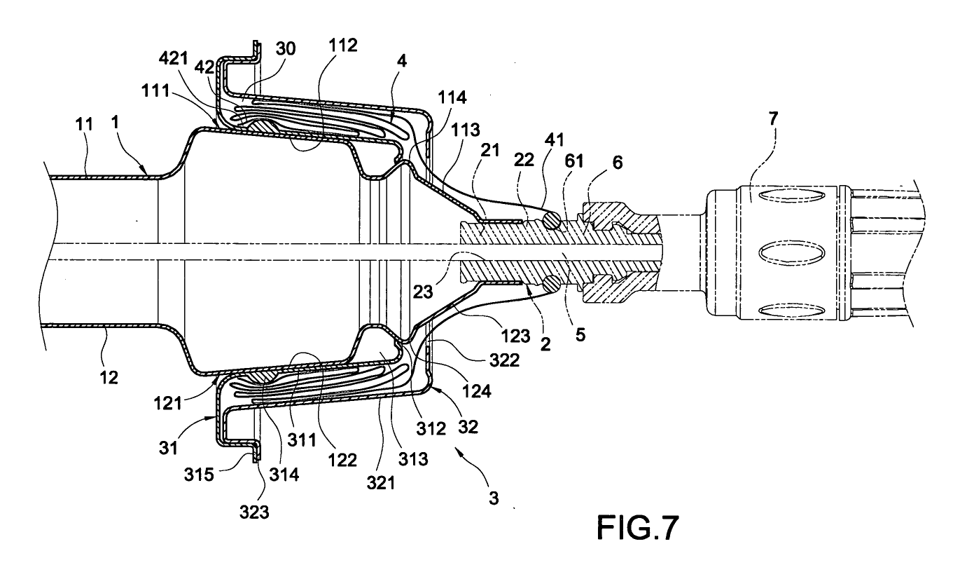

Please refer to FIG. 7, which is an illustration of another embodiment of the present invention. As shown in this figure, the disclosed sheath structure is roughly same as that shown in FIG. 1 to FIG. 8. The difference is that the hole 411 of the first side 41 of the sheath 4 is connected an elastic element 43 of ring type, which is made of rubber material.

During the application of the sheath 4, it is possible to tie the elastic element 43 directly on the ring groove 62 of the adapter 6, pushing the container 3 toward the grip structure 7 of the portable medical equipment. After the container 3 is separated from the first snapping parts 111, 121 and the second snapping parts 113, 123 at the front side of the sheath 1, the sheath 4 accommodated between the first bulger 311 of the first cup 31 and the second bulger 321 of the second cup is released in sequence and covers the entire grip structure 7, keeping the grip structure 7 from being stained with patient's blood or any bacteria, when a medical care or operation is undertaken.

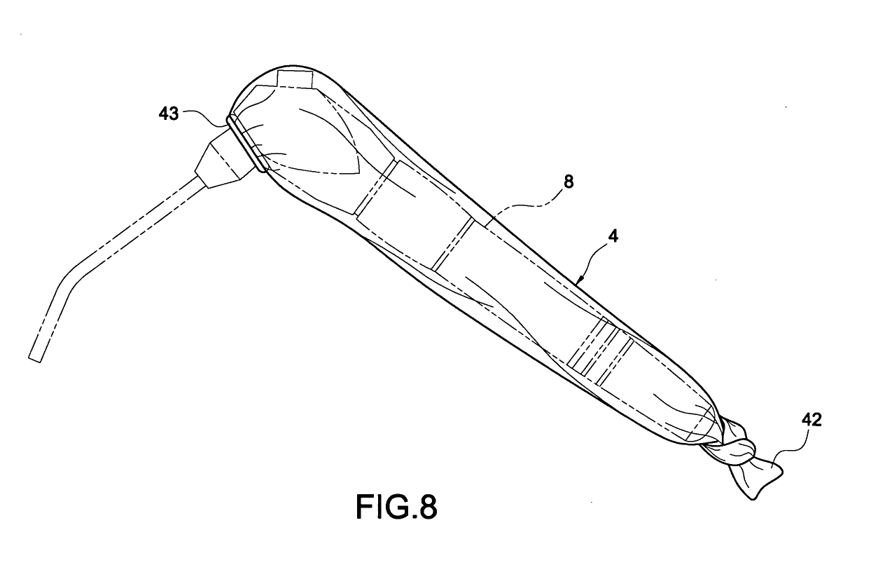

Please refer to FIG. 8, which is an illustration of a further embodiment of the present invention. As shown in this figure, it is possible to fit the sheath 4 of FIG. 7 onto another kind of portable medical equipment 8. First of all, the elastic element 43 of the sheath 4 is tied onto the portable medical equipment 8. Then, the sheath 4 is fitted over the portable medical equipment 8 and covers it allover. Thus, it is possible to keep the portable medical equipment 8 from being stained with patient's blood or any bacteria, when a medical care or operation is undertaken.

Summarizing aforementioned description, the invention is an indispensable product of novelty indeed, which may positively reach the expected usage objective for solving the drawbacks of the prior arts, and which extremely possesses the innovation and progressiveness for completely fulfilling the applying merits of new type patent, according to which the invention is thereby applied. Please examine the application carefully and grant it as a formal patent for protecting the rights of the inventor.

However, the aforementioned description is only a number of preferable embodiments according to the present invention, being not used to limit the patent scope of the invention, so equivalently structural variation made to the contents of the present invention, for example, description and drawings, is all covered by the claims claimed thereinafter.

What is claimed is:

1. A sheath structure for a portable medical equipment, assembled to an adapter of the medical equipment and fitted onto the medical equipment, including:
a snap-on body assembled to the adapter, having a main body, a catch being arranged at one side of the main body, a perforation penetrating the main body, and a ring groove being arranged between the perforation and the catch so as to be assembled and connected to a projecting ring of the adapter;
a container comprised of a first cup and a second cup, the first cup having a first bulger, a first opening being arranged at one side of the first bulger, and a first projecting wall being extended at another side; in addition, the second cup having a second bulger with an inner diameter larger than an outer diameter of the first bulger, a second opening being arranged on one side face of the second bulger, and a second projecting wall being arranged at another side; the second projecting wall of the second cup being embedded into the first cup between an inner wall surface of the first projecting wall and the first bulger, making an accommodating space formed between an outer surface of the first bulger and an inner wall surface of the second bulger; and
a sheath, folded in the accommodating space and having a first side and a second side, a hole disposed in the ring groove being arranged on one side face of the first side, an side face of the second side being shown as a third opening.

2. The sheath structure of portable medical equipment according to claim 1, wherein the snap-on body is shown as an “I” shape.

3. The sheath structure of portable medical equipment according to claim 1, wherein another catch is arranged at another side of the main body of the snap-on body.

4. The sheath structure of portable medical equipment according to claim 1, further including a light source transmitting body arranged through the perforation of the snap-on body.

5. The sheath structure of portable medical equipment according to claim 1, wherein the first and second bulgers are shown as a hollow shape.

6. The sheath structure of portable medical equipment according to claim 1, wherein a snapping side curved inwardly is arranged at one side of the first bulger.

7. The sheath structure of portable medical equipment according to claim 1, wherein a projecting rib ring is arranged on an outer surface of the first bulger.

8. The sheath structure of portable medical equipment according to claim 1, wherein the first projecting wall is folded outwardly from one side of the first bulger.

9. The sheath structure of portable medical equipment according to claim 1, wherein the second projecting wall is folded outwardly from one side of the second bulger.

10. The sheath structure of portable medical equipment according to claim 1, wherein the sheath is a thin film envelope made of transparent plastic.

11. The sheath structure of portable medical equipment according to claim 1, further including a protecting shell assembled to the snap-on body and comprised of two shell bodies corresponding to each other, two projecting first snapping parts being arranged at one side of the two shell bodies, an arc snapping face being arranged on the surface of each two first snapping parts.

12. The sheath structure of portable medical equipment according to claim 11, wherein two second snapping parts shown as a cone shape are respectively connected to the front sides of two first snapping parts, two snapping ribs being arranged on two second snapping parts for snapping on the snapping side of the first bulger.

13. The sheath structure of portable medical equipment according to claim 12, wherein a jointing hole communicating an interior of the sheath is arranged at the front side of two combined second snapping parts for jointing the main body of the snap-on body.

14. The sheath structure of portable medical equipment according to claim 11, wherein the sheath is made of materials of transparent plastic.

15. A sheath structure for a portable medical equipment, assembled with a ring groove in an adapter of the medical equipment and fitted onto the medical equipment, including:
a container comprised of a first cup and a second cup, the first cup having a first bulger, a first opening being arranged at one side of the first bulger, and a first projecting wall being extended at another side of the first bulger; in addition, the second cup having a second bulger with an inner diameter larger than an outer diameter of the first bulger, a second opening being arranged on one side face of the second bulger, and a second projecting wall being arranged at the another side of the second bulger; the second projecting wall of the second cup being embedded into the first cup between an inner wall surface of the first projecting wall and the first bulger, making an accommodating space formed between an outer surface of the first bulger and an inner wall surface of the second bulger; and
a sheath, folded in the accommodating space and having a first side and a second side, a hole disposed in the ring groove being arranged on one side face of the first side, an side face of the second side being shown as a third opening.

16. The sheath structure of portable medical equipment according to claim 15, wherein the first and second bulgers are shown as a hollow shape.

17. The sheath structure of portable medical equipment according to claim 15, wherein a snapping side curved inwardly is arranged at one side of the first bulger.

18. The sheath structure of portable medical equipment according to claim 15, wherein a projecting rib ring is arranged on an outer surface of the first bulger.

19. The sheath structure of portable medical equipment according to claim 15, wherein the first projecting wall is folded outwardly from one side of the first bulger.

20. The sheath structure of portable medical equipment according to claim 15, wherein the second projecting wall is folded outwardly from one side of the second bulger.

21. The sheath structure of portable medical equipment according to claim 15, wherein the sheath is a thin film envelope made of transparent plastic.

22. The sheath structure of portable medical equipment according to claim 15, wherein the elastic element is shown as a ring shape.

23. The sheath structure of portable medical equipment according to claim 15, wherein the elastic element is made of rubber materials.
